(19) United States
(12) Patent Application Publication
(10) Pub. No.: US 2014/0051105 A1
(43) Pub. Date: Feb. 20, 2014

(54) Mutant Proteins as Cancer-Specific Biomarkers

(71) Applicants: Bert Vogelstein, Baltimore, MD (US); Qing Wang, Parkville, MD (US); Akhilesh Pandey, Ellicott city, MD (US); Kenneth W. Kinzler, Baltimore, MD (US); Nickolas Papadopoulos, Towson, MD (US)

(72) Inventors: Bert Vogelstein, Baltimore, MD (US); Qing Wang, Parkville, MD (US); Akhilesh Pandey, Ellicott city, MD (US); Kenneth W. Kinzler, Baltimore, MD (US); Nickolas Papadopoulos, Towson, MD (US)

(73) Assignee: THE JOHNS HOPKINS UNIVERSITY, Baltimore, MD (US)

(21) Appl. No.: 13/980,005

(22) PCT Filed: Jan. 17, 2012
(86) PCT No.: PCT/US2012/021553

§ 371 Date: Oct. 29, 2013

Related U.S. Application Data
(60) Provisional application No. 61/433,376, filed on Jan. 17, 2011.

(51) Int. Cl. G01N 33/68

(52) U.S. Cl. CPC G01N 33/6893
USPC 435/23

(57) ABSTRACT

Altered protein products resulting from somatic mutations are directly identified and quantified by mass spectrometry. The peptides expressed from normal and mutant alleles are detected by Selected Reaction Monitoring (SRM) of their productions using a triple quadrupole mass spectrometer. As a prototypical example of this approach, we quantify the number and fraction of mutant Ras protein present in cancer cell lines. There were an average of 1.3 million molecules of Ras protein per cell and the ratio of mutant to normal Ras proteins ranged from 0.49 to 5.6. Similarly, we detected and quantified mutant Ras proteins in clinical specimens such as colorectal and pancreatic tumor tissues as well as in pre-malignant pancreatic cyst fluids. These methods are useful for diagnostic applications.

This invention was made using funds from the U.S. Government. The U.S. Government retains certain rights in the invention according to the provisions of grants from the National Institutes of Health CA 43460, NO1 CN-43302, and CA 62924.

### TECHNICAL FIELD OF THE INVENTION

This invention is related to the area of protein detection. In particular, it relates to quantification and identification of proteins present in complex mixtures.

### BACKGROUND OF THE INVENTION

Through genome-wide analysis, it has recently been shown that solid tumors typically contain 20 to 100 protein-encoding genes that are mutated (1-4). A small fraction of these changes are “drivers” that are responsible for the initiation or progression of the tumors, while the remainder are “passengers”, providing no selective growth advantage (5, 6). In principle, these proteins provide unparalleled opportunities for biomarker development. Unlike other protein biomarkers such as CEA or PSA, the mutant proteins are only produced by tumor cells. Moreover, they are not simply associated with tumors, as are conventional markers, but in the case of driver gene mutations, they are directly responsible for tumor generation.

The detection of the proteins encoded by mutated genes (henceforth termed “mutant proteins”) is straightforward when proteins are truncated by a nonsense mutation or fused to other proteins. This can often be accomplished simply by Western blotting of cellular extracts. However, the majority of disease-causing mutations are missense mutations that only subtly alter the encoded proteins. For example, in recent studies of the sequences of all protein-encoding genes in human cancers, >80% of the somatic mutations were reported to be missense (1-3). Although it is theoretically possible to directly detect these abnormal proteins with antibodies directed against mutant epitopes, this has been difficult to accomplish in practice. For example, though KRAS and TP53 are two of the most commonly mutated and intensely studied cancer genes, there are still no antibodies that can reliably distinguish mutant from normal versions of these proteins. The fact that many different mutations can occur in a single cancer-related gene makes it necessary to develop a specific antibody for each possible mutant epitope, compounding the difficulty of success achievable through this strategy. Another approach employs measurement of the activity of mutant proteins. Though this can be useful in special situations, it is not generally applicable because there are no activity-based assays available for most proteins and the proteins resulting from mutated genes often have activities that are only quantitatively, rather than qualitatively, different from their normal counterparts. There is thus a critical need for developing assays that would permit quantification of mutant proteins in a generic fashion.

Recent advances in mass spectrometry (MS) permit sampling of a large fraction of normal and abnormal cellular proteomes in an unbiased and specific fashion (7, 8). MS has already become the method of choice to quantify protein levels and a number of quantitative proteomics strategies for this purpose have been described (9-14). Interestingly, mass spectrometry has already been used to detect and precisely quantify somatic mutations—but at the DNA level—not at the protein level (15). Indeed, one of the most widely-used methods for quantifying such mutations in DNA relies on the measurement of the mass of oligonucleotides differing at a single base (16). Prior studies have shown that it is possible to identify post-translationally altered proteins using MS, as well as to identify highly abundant abnormal proteins, such as those responsible for amyloidosis (17-22). In this work, we have sought to develop a mass spectrometric approach that could be used to identify and quantify somatically mutant proteins in a generally applicable fashion. We were particularly interested in working out a strategy that could be applied to complex biological samples such as those encountered clinically.

There is a continuing need in the art to identify and quantify mutant proteins in complex clinical samples.

### SUMMARY OF THE INVENTION

An aspect of the invention is a method of detecting the presence or amount of a mutant form of a selected protein in a biological sample. The selected protein is enriched in the biological sample to form an enriched sample. The selected protein in the enriched sample is fragmented using a site-specific endoprotease to form a fragmented, enriched sample comprising a selected peptide. The fragmented, enriched sample is spiked with a known amount of a heavy-isotope labeled form of the selected peptide. The spiked fragmented, enriched sample is subjected to liquid chromatography to form output fractions having distinct peptide profiles. The output fractions are directed to a triple quadrupole mass spectrometer to form product ions. Selected product ions of the selected peptide representing wild type and/or mutant forms of the selected protein and product ions of the heavy-isotope labeled form of the selected peptide are detected.

These and other aspects which will be apparent to those of skill in the art upon reading the specification provide the art with powerful techniques for analyzing clinical samples for mutations.

### BRIEF DESCRIPTION OF THE DRAWINGS

FIG. 1 shows a schematic drawing of the overall approach to analyzing biological samples.

FIG. 2 shows immunoprecipitations of Ras proteins. An antibody directed against a common epitope of all three forms of mutant and WT forms of Ras (K-Ras, N-Ras, and H-Ras) was used to immunoprecipitate the indicated amounts of protein in SW480 cell lysates. Western blots were performed using a horseradish peroxidate-conjugated monoclonal antibody to K-Ras. Ten ng of recombinant K-Ras protein was loaded on the right-most lane of each gel for comparison purposes. The “input lysate” and “lysate after IP” lanes contained 4% of the proteins used for IP, while all of the “eluted protein” and protein “remaining on beads” were loaded into the corresponding lanes.

FIG. 3A-3F shows extracted ion chromatograms of ¹³C/¹⁵N-labeled synthetic peptides. The retention times of the indicated peptides are shown above the peaks in (A-C; SEQ ID NO: 1, 5, and 6, respectively), and the insets at the right of each figure represent an expanded view. The asterisks (*) indicate the heavy-isotope (¹³C₆¹⁵N₂) labeled lysine. FIGS. 3D (SEQ ID NO: 1), 3E (SEQ ID NO: 5) and 3F (SEQ ID NO: 6) illustrate the relationship between the amount of peptides injected into the mass spectrometer and the integrated intensity of the transitions. The b and y peaks indicate the detected intensities of b and y ions (as designated in Table 2 (S1)).

FIG. 4A-4D shows SRM of endogenous proteins from SW480 cells. (FIG. 4A; SEQ ID NO:1) Extracted ion chromatograms of transitions from the exogenously added heavy-isotope labeled WT peptide and corresponding endogenous WT peptide (FIG. 4B; SEQ ID NO:1), illustrating the identical retention times. (FIG. 4C; SEQ ID NO:6, FIG. 4D; SEQ ID NO:6) Extracted ion chromatograms of the exogenous and endogenous mutant peptides, respectively. In each case, the inset at the right represents an expanded view of the major peaks. The asterisks (*) indicate the heavy isotope (¹³C₆¹⁵N₂) labeled lysine.

FIG. 5A-5B shows SRM of endogenous proteins from a colorectal tumor obtained at surgery. (FIG. 5A; SEQ ID NO:5) Integrated intensities of the exogenously added, mutant peptide and the endogenous mutant peptide from the tumor, as indicated. The integrated intensities correspond to the sum of the peak areas of the transitions described in Table 2 (S1), which are shown in (FIG. 5B; SEQ ID NO:5) for the endogenous peptide. The asterisk (*) indicate the heavy isotope (¹³C₆ ¹⁵N₂) labeled lysine.

FIG. 6 (S1). Trypsin digestion maps of the first 100 residues of K-Ras(SEQ ID NO:1 and 2, respectively), N-Ras (SEQ ID NO:1 and 3, respectively) and H-Ras (SEQ ID NO:1 and 4, respectively) proteins.

FIG. 7 (S2). Correlations between input amounts of lysate and WT and mutant Ras peptides detected by SRM. The endogenous WT and G12V mutant Ras peptides were quantified by comparison with the exogenously added heavy-isotope labeled synthetic peptides.

FIG. 8 (S3). Determination of peptide loss during the SRM procedure. 50 to 2000 ng (corresponding to 1 to 43 pmole of the GST tagged recombinant K-Ras protein, MW: 46.4 kDa) of K-Ras recombinant protein was spiked into SW480 cell lysates each containing 2 mg of total cellular protein, and SRM was performed. The y-axis represents the calculated amount of peptide observed in the MS after subtraction of the 1.6 pmoles contributed by the endogeous WT Ras proteins present in SW480 cells. The recovery was determined from the slope of the trend line to be 22.4%.

FIG. 9 (S4). Chromatograms of peptides derived from K-Ras (SEQ ID NO:2), N-Ras (SEQ ID NO:3), and H-Ras (SEQ ID NO:4) proteins derived from SW480 cells. The transitions of the indicated peptides are described in Table 2 (S1).

FIG. 10 (S5). Confirmation of peptides used for SRM-based quantification. (A-C) MS/MS spectra of the indicated peptides from wild type Ras (FIG. 10A; SEQ ID NO:1), mutant Ras (FIG. 10B; SEQ ID NO:5) and N-Ras (FIG. 10C; SEQ ID NO:3) proteins of Pal6c cells. (FIG. 10D- FIG. 10G) MS/MS spectra of the indicated peptides from wild type Ras (FIG. 10D; SEQ ID NO:1), mutant Ras (FIG. 10E; SEQ ID NO:6), K-Ras (FIG. 10F; SEQ ID NO:2) and N-Ras (FIG. 10G; SEQ ID NO:3) proteins from SW480 cell line. The transitions of the indicated peptides are described in Table 2 (S1).

### Table Legends

FIG. 11. Table 1. Levels of WT (SEQ ID NO:1) and mutant Ras proteins (SEQ ID NO:6, 5, and 7, respectively) in cells and tissues (pmoles/2 mg cellular protein).

FIG. 12. Table 2 (S1). SRM Transition Parameters. Peptide sequences shown are SEQ ID NO: 1, 6, 5, 2, 3, and 4, respectively.

FIG. 13. Table 3 (S2). Relative levels of K-Ras (SEQ ID NO:2), N-Ras (SEQ ID NO:3), H-Ras (SEQ ID NO:4) proteins

### DETAILED DESCRIPTION OF THE INVENTION

The inventors have developed a two-component system for the detection of minute quantities of proteins which is useful for analysis of clinical specimens which are biochemically complex. The system comprises an initial enrichment of the protein of interest and then a targeted analysis of peptides derived from this protein. Additional components can be used in conjunction for particular applications.

The approach described here fulfills a heretofore unmet need in cancer research, diagnosis, monitoring, and theranostics, permitting the determination of the relative amounts of missense mutant and wild-type (WT) proteins and allowing comparisons among the amounts of DNA, RNA, and polypeptides. The determination of the relative levels of mutant and WT proteins can help inform the mechanisms underlying the abnormal protein's function, e.g., through supporting the basis for dominant-negative effects or haploinsufficiency. The approach opens up new diagnostic and prognostic opportunities, as illustrated by the results described below on pancreatic cysts. One advantage of protein based analysis over DNA-based approaches is that numerous independent proteins can be assessed simultaneously, thereby preserving precious clinical samples and reducing the costs of clinical analyses. Another advantage is that no amplification is needed, thereby minimizing the contamination issues that often plague PCR-based approaches (35).

Enrichment of a desired protein target can be accomplished by any means known in the art. A host of enrichment procedures are available, including but not limited to precipitation, chromatography, electrophoresis, solvent partitioning, immunoprecipitation, immunoelectrophoresis, and immunochromatography. Any can be used to achieve an enrichment of the protein of interest. One method employs antibodies to immunoprecipitate the desired protein target. The antibodies can be attached, optionally, to a solid support such as a bead, magnetic bead, or other solid particle. One means of attachment is conjugation of the antibody to a protein coated on the beads. Other means of attachment can be used, such as direct coating of a bead with the antibody. After separation of the antibody bound protein from free proteins, the bound protein can be eluted. Any elution means can be used. One elution means which has been found to be efficient is 3% acetic acid. Other elutions means, including other acids, and other concentrations of acetic acid can be used, as is efficient for a particular protein.

The enriched protein can be subjected to a fragmentation procedure to produce a defined set of protein fragments. This can be readily accomplished using site specific endoproteases, such as pepsin, arg-C proteinase, asp-N endopeptidase, BNPS-skatole, caspase 1, caspase 2, caspase 3, caspase 4, caspase 5, caspase 6, caspase 7, caspase 8, caspase 9, caspase 10, chymotrypsin, clostripain (clostridiopeptidase B), enterokinase, factor Xa, glutamyl endopeptidase, granzyme B, lysC, proline-endopeptidase, proteinase K, staphylococcal peptidase I, thermolysin, thrombin, and trypsin. Chemicals which cleave site specifically can also be used. Combinations of enzymes and/or chemicals can be used to obtain desirable analytes.

In order to obtain an absolute value of mutant peptide, a known amount of a synthetically produced version of a selected peptide produced by the fragmentation procedure is added to the fragmented sample. The synthetic peptide is labeled with a heavy isotope so that it is distinguishable from the endogenous peptide produced by the fragmentation of the sample. Conveniently, the peptide is labeled with C¹³/N¹⁵ heavy isotopes. Other isotopes can be used alternatively.

The fragments can be directed to the triple quadrupole instrument using electrospray or Matrix-assisted laser desorption/ionization (MALDI), for example. These generate ionized versions of the fragments. Other techniques which may be used include Chemical ionization (CI), Plasma and glow discharge, Electron impact (EI), Fast-atom bombardment (FAB), Field ionization, Laser ionization (LIMS), Plasma-desorption ionization (PD), Resonance ionization (RIMS), Secondary ionization (SIMS), Spark source, and Thermal ionization (TIMS).

Fragments or transitions for monitoring are chosen for analysis. Chromatograms of wild type and mutant proteins, heavy isotope labeled and endogenous, are used for quantification of the different forms of the protein. We found that that the ratio of mutant to wild type is independent of the amount of input protein.

Clinical or biological samples which can be subjected to this method are not limited. The sample may derive from human, plant, other mammal or animal, bacterial, or fungal sources, for example. The sample may be from a single individual or from a population of individuals. The sample can be from a solid tissue obtained from an in vivo source, from a biological fluid, such as urine, sputum, blood, lymph, stool, exudate, breast milk, cyst liquid, etc. The sample may be from a culture medium of cells grown in vitro. The sample may comprise neoplastic cells, proteins from neoplastic cells, pre-malignant cells, proteins from pre-malignant cells, etc.

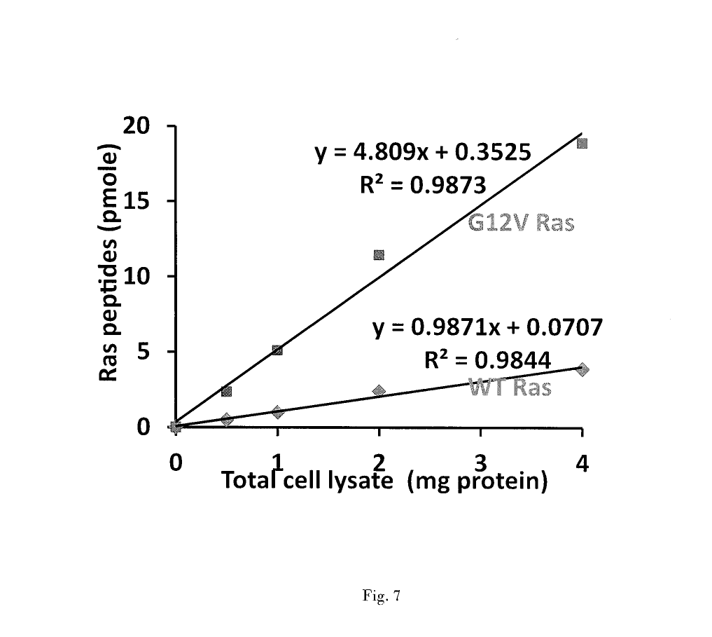

The results described below show that selected reaction monitoring (SRM) can be used to detect and quantify the levels of WT and mutant proteins in cell lines as well as in clinically-relevant tissue samples and biologic fluids. This approach is the only one so far described that can generally be used for this purpose. Several advantages are apparent from the data: the technique is sensitive, allowing detection of as little as 10 fmole; the calculated levels of WT and mutant proteins are linearly related to input over a wide range (FIG. 7 (S2)); the use of internal controls and the monitoring of multiple product ions ensure exquisite specificity; and the technique is relatively simple to implement. It can be implemented with commercially available reagents, such as an antibody against the normal form of the protein and a state-of-the-art mass spectrometer. In particular, it does not require the development of antibodies that are mutant-specific, which can be difficult, especially when many antibodies would be required to target proteins that have multiple mutant forms.

We estimate that the method can be used to reliably detect mutant proteins when they are present at levels as low as 25 fmole in 1 mg of total protein. We could thus detect mutant and WT Ras proteins in as few as 6000 cells. However, increased sensitivity may be required to detect mutant proteins in some clinical samples, such as sputum, serum, or urine. Success of detection can be increased by increasing the amount of sample used for enrichment. Further improvements in mass spectrometer instrumentation can be expected to improve this sensitivity. Additionally, various steps involved prior to MS—pulverization, homogenization, immunoprecipitation, elution, trypsinization, and chromatography—can be improved to reduce sample loss. Such improvements can permit detection of an analyte in as few as 3000, 1000, 500 or 300 cells, and further improvements are possible.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLES

Although MS-based technologies are capable of detecting attomole minute quantities of proteins (23), their sensitivity can be compromised by many factors, including sample preparation and the biochemical complexity of clinical specimens (24). For this reason, the work described here involved the implementation of two independent components: enrichment of the protein of interest and the targeted analysis of peptides derived from this protein.

### EXAMPLE 1

### Materials and Methods

Materials. The SW480 colorectal cancer cells were purchased from ATCC (Rockville, Md.). The Pa02C, Pa08C, and Pa16C pancreatic cancer cell lines were derived as described (36). Colorectal tumors and cyst fluids were obtained from surgical resection specimens at the Johns Hopkins Hospital. Tissues and cyst fluids were flash frozen within 30 minutes of excision and stored at −80° C. All samples were obtained in accordance with the Health Insurance Portability and Accountability Act (HIPAA) and had Institutional Review Board approval.

A rabbit monoclonal [EP1125Y] antibody reactive with all three Ras isoforms (K-Ras, N-Ras, and H-Ras; Cat no. ab52939] was purchased from Abcam (Cambridge, Mass.). A mouse monoclonal antibody specific to K-Ras [Cat#: SC-30] was purchased from Santa Cruz Biotechnology (Santa Cruz, Calif.). All other reagents were purchased from Sigma-Aldrich unless otherwise indicated.

Antibody conjugation reaction buffer (ACRB): 0.2 M triethanolamine, pH8.2, 20 nM dimethyl pimelimidate dihydrochloride. Prepared fresh before each use.

Lysis buffer (10 ml): 6.87 ml of RIPA buffer (68.7 μl NP-40, 687 μl of 10% sodium deoxycholate, 68.7 μl of 10% SDS (Invitrogen; Carlsbad, Calif.), 206.1 μl of 5 M NaCl, 68.7 μl of 1 M Sodium phosphate, pH 7.2, 1 ml water, one Complete EDTA-free Protease Inhibitor Cocktail Tablet (Roche; Indianapolis, Ind.), 1000 μl 0.5 M NaF, 10 μl of 80 mM b-glycerophosphate, 1000 μl of 20 mM Na pyrophosphate, 10 μl of 300 mM Na orthovanadate, 10 μl of 1M DTT, 100 μl of 100 mM PMSF.

Modified RIPA Buffer (10 ml): 300 μl of 5 M NaCl, 500 μl of 1M Tris, pH 7.4, 100 μl NP-40, 250 μl of 10% sodium deoxycholate, 20 μl of 0.5 M EDTA, water 8.83 ml.

Mass Spectrometry solvents: Solvent A: 3% Acetonitrile, 0.1% Formic Acid; Solvent B: 90% Acetonitrile, 0.1% Formic Acid.

Immobilization of antibody on magnetic beads. Conjugation of antibodies to beads was performed using slight modifications of methods described by Whiteaker et al. (26). The rabbit monoclonal antibody to Ras (100 μl) was added to 500 μl Protein G Dynal Magnetic Beads (directly obtained from Invitrogen, without further washing) and the antibody was bound to the beads on a rotator at room temperature for 1 h. The antibody-bound beads were then washed by incubation in 1 ml ACRB and collected on a magnet. To cross-link the antibody to the protein G on the beads, they were then incubated with 1 ml of ACRB on a rotator at room temperature for 30 min. The beads were then washed twice with 1 ml 50 mM Tris-HCl (pH 7.5), then resuspended in 1 ml 50 mM Tris-HCl (pH 7.5) and rotated at room temperature for 15 min. The incubation with Tris-HCl stopped the cross-linking reaction. The beads were finally resuspended in 300 gl 50 mM Tris-HCl (pH 7.5) and 200 gl glycerol and stored at −20° C.

Cell lysis and protein quantification. Cultured cells were lysed by incubation in Lysis Buffer for 30 min on ice, vortexing every 10 min. Tissues were lysed by placing 50 mg into a Covaris tube (Covaris; Woburn, Massachusetts), which was then frozen in liquid nitrogen, and pulverized twice using a Covaris CP02 instrument (Covaris; Woburn, Massachusetts). The frozen tissue powder was transferred to Covaris glass vials (Covaris; Woburn, Mass.), 1 ml of Lysis Buffer was added, and the suspension was sonicated once every 15 minutes for a total of four times using a Covaris S2 instrument (Covaris; Woburn, Mass.) with the following settings:
Step 1: Duty cycle 5%, Intensity 3%, Cycles/Burst 100, 5 s;
Step 2: Duty cycle 20%, Intensity 8%, Cycles/Burst 100, 30 s;
Step 3: Duty cycle 5%, Intensity 3%, Cycles/Burst 100, 5 s.
Step 4: Duty cycle 5%, Intensity 3%, Cycles/Burst 100, 5 s (this is a repeat of Step 3).

The homogenate was kept on ice between sonications. The lysates from cells or tissues were clarified by centrifugation at 12,000 g for 30 min at 4° C. Lysates were stored at −80° C., 2 mg of cellular protein per tube. A BCA assay kit (Thermo; Rockford, Ill.) was used to quantify protein concentrations.

Immunoprecipitation of Ras proteins. Cell lysates containing 2 mg of total protein (generally ˜200 μl) was thawed on ice and diluted with 4 volumes of Modified RIPA buffer. Antibody-conjugated Dynal beads (100 μl) were added and the suspension incubated at 4° C. overnight (minimum of 12 hr). The beads were collected on a magnet, washed 3 times with freshly prepared modified RIPA buffer. The bound Ras proteins were eluted by vortexing the beads at 650 RPM in 100 μl 3% Acetic acid for 30 min at 37° C. on a Thermomixer (Eppendorf; Hamburg, Germany). The solution was neutralized by adding 2 volumes of 1 M ammonium bicarbonate.

Trypsin digestion. 450 pl methanol were added to 300 μl of the neutralized Ras protein eluate, giving a final concentration of 60% methanol. DTT was added to a final concentration of 1 mM and the solution was incubated at 60° C. on a Thermomixer for one hr at 650 RPM. The solution was cooled to room temperature and iodoacetamide was added to a final concentration of 50 mM, and then incubated at room temperature in the dark for 30 min. 3.25 ml Distilled water was added to dilute the ammonium bicarbonate to 50 mM. The pH of the solution was ˜8.0. Sequencing grade trypsin (Promega) was added to a final concentration of 5 μg/ml and incubated at 37° C. overnight. The peptide solution was then acidified by adding 1% trifluoroacetic acid (TFA) and incubated at RT for 15 min. A Sep-Pak light C₁₈ cartridge (Waters; Milford, Mass.) is activated by loading 5 ml 100% acetonitrile, and washed by 3.5 ml 0.1% TFA solution 2 times. Acidified digested peptide solution was centrifuged at 3,000 rpm and the supernatant was loaded into the cartridge. One ml, 3 ml and 4 ml of 0.1% TFA were sequentially used to desalt the peptides bound to the cartridge. Two ml of 40% acetonitrile with 0.1% TFA was used to elute the peptides from the cartridge and this elution was repeated two more times (for a total of 6 ml of eluate). It was important to ensure that the cartridge had stopped dripping before each sequential wash and elution solution was applied. The eluted peptides were lyophilized overnight and re-dissolved in 40 μl A of Solution A.

HPLC. Peptide samples were separated using a reversed phase column (XBridge BEH130 C₁₈ Column, 5 μm, 2.1×250 mm) (Waters; Milford, Mass.) on the 1200 LC system (Agilent Technologies, Santa Clara, Calif.). After loading 40 μl of peptide sample into the column, the LC gradient was generated in 0.1% formic acid with increasing acetonitrile concentrations using gradient solvent B from 0 to 3% for the first 6 min, then 3 to 10% for 4 minutes, and 10 to 40% for the subsequent 20 minutes. The column was regenerated by continuing the gradient up to 100% solvent B for the next 6 minutes, then reversing the gradient from 100% to 3% solvent B over the next 2 minutes, and finally equilibrating in 3% solvent B for 8 minutes. A saw-tooth gradient consisting of alternating increases and decreases in solvent B concentration (0-100% and 100-0% for 10 min, repeated twice for a total of 3 times) was used to prevent carryover of the peptides. A blank sample (no protein) was then loaded into the LC and subjected to the gradient described above before the next experimental sample was loaded.

Mass spectrometry. Drying gas: 12 L/min, 300° C.; Fragmentor: 130 V; Dwell time: 10 ms; capillary voltage: 4,000 V; Resolution of Q1 and Q3: unit mass; collision energy: optimized for each peptide (Table 2 (51)) with the Agilent MassHunter Peptide Optimizer. One pmole of synthetic peptides with ¹³C/¹⁵N-labeled arginine or ¹³C/¹⁵N-labeled lysine at its C-terminus (Sigma; St. Louis, Mo.) were used for optimization of transition parameters. SRM analysis was carried out in positive mode using a 6460 Triple Quadrupole Mass Spectrometer (Agilent Technologies, Santa Clara, Calif.) equipped with a capillary flow (100 μl per min) ESI connected to the 1200 capillary pump.

SRM Data analysis. A list of transitions were selected based on MassHunter Peptide Optimizer data for each heavy-isotope labeled peptide (¹³C₆¹⁵N₂ lysine and ¹³C₆¹⁵N₄ arginine). The peaks of each y ion and b ion that could be generated from peptides with 2+ and 3+ charge states were optimized by altering the collision energy for each transition. The Skyline program (37) preloaded with WT and mutant Ras peptide sequences was used to analyze the data. The endogenous peptide-specific peaks were identified by comparison to the exogenously added ¹³C/¹⁵N-labeled peptides, which were 8 Da and 10 Da heavier for lysine and arginine containing peptides respectively. In addition, the retention times and transition profiles of the exogenous and endogenous peptides were manually inspected to ensure that they were internally consistent. Peptide abundance was calculated from integrating the areas representing the peaks of each detected exogenous and endogenous ion. Each analysis described in the text or listed in Table 1 or Table 3 (S2) was repeated at least once, and averages and standard deviations are reported.

Full Scan LC-MS/MS and Data Analysis. The tryptic digested peptides from immuoaffinity enriched proteins were purified on a strong cation exchange stage-tip using binding and washing buffer 5 mM KH₂PO₄ pH 2.7 25% Acetonitrile and an elution buffer containing 1% ammonium hydroxide in 25% acetonitrile. LC-MS/MS analysis of dried peptides was carried out using a chip cube interfaced to a UHD Accurate-Mass QTOF LC/MS (Agilent Technologies, Santa Clara, Calif.). The chip LC system consisted of 160 nl peptide enrichment column and a 150 mm analytical column packed with Zorbax 300 SB C₁₈, 5 μm reversed phase material. The peptides were separated by acetronitrile gradient (10-35%) containing 0.1% formic acid. The MS/MS spectra were acquired in a data-dependent manner, targeting the four most abundant ions in each survey scan from 350-1,700 m/z range and MS/MS scan from 100-1,700 m/z range using a collision energy set-up of 3.0 V/100 Da, Offset 2 V. Dynamic exclusion was enabled after acquisition of 2 spectra for 15 seconds. The data were searched using Spectrum Mill software against human RefSeq database version 40 containing 31,789 protein sequences appended with different mutant Ras protein sequences. Carabmidomethylation was allowed as fixed modification and oxidation of M and deamidation N and Q were permitted as variable modifications. One missed cleavage was allowed for searching tryptic peptides. Mass tolerances of 20 ppm and 50 ppm were allowed for MS and MS/MS spectra identification.

### Example 2

### Enrichment of Proteins For SRM Experiments

Among the available methods for enrichment of proteins, we chose immunoprecipitation (IP) for several reasons. First, antibodies have been generated against most proteins of interest and SRM does not require the antibodies to be absolutely specific for the antigens or specific for the mutations of interest; this specificity comes from the subsequent MS analysis. Second, immunoprecipitation removes the most abundant proteins from biological samples, including cytoskeletal proteins, immunoglobulins, and serum albumin (25, 26). And third, it is scalable and can be readily applied to samples containing large volumes or high concentrations of irrelevant proteins.

We used cancer cells in culture to optimize the immunoprecipitation methods, with K-Ras as the target. The KRAS gene is commonly mutated in human colorectal and pancreatic cancers, with most mutations clustered at residues 12 or 13 of the protein. Several methods for lysing cells and capturing Ras proteins were explored in order to obtain the great majority of the Ras protein in a form compatible with subsequent MS analysis. We found that cell lysis in a detergent-containing buffer followed by binding to antibody-coupled magnetic beads, achieved these goals (25 and see Materials and Methods). Covalent coupling of the antibody to magnetic beads was performed using dimethyl pimelimidate (DMP). After binding of the antigen to the immobilized antibodies, Ras was eluted and concentrated. Of the elution methods tried (various concentrations of acids, bases, glycine, detergents, and denaturants at various temperatures and times), we found that 3% acetic acid most reproducibly eluted Ras proteins in a fashion that facilitated subsequent protease digestion.

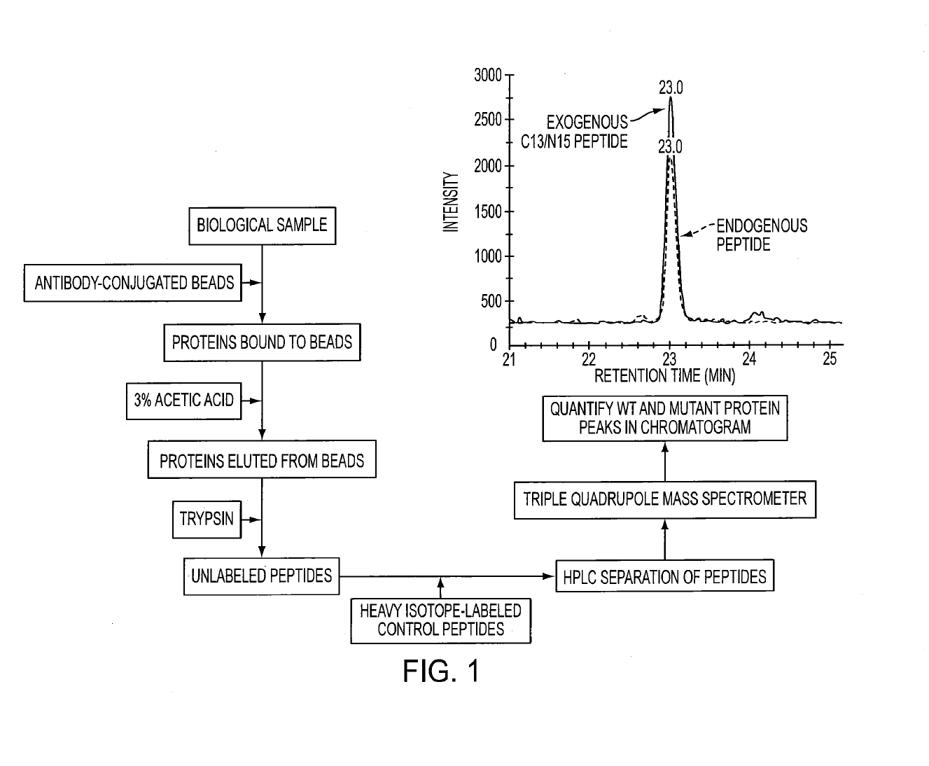

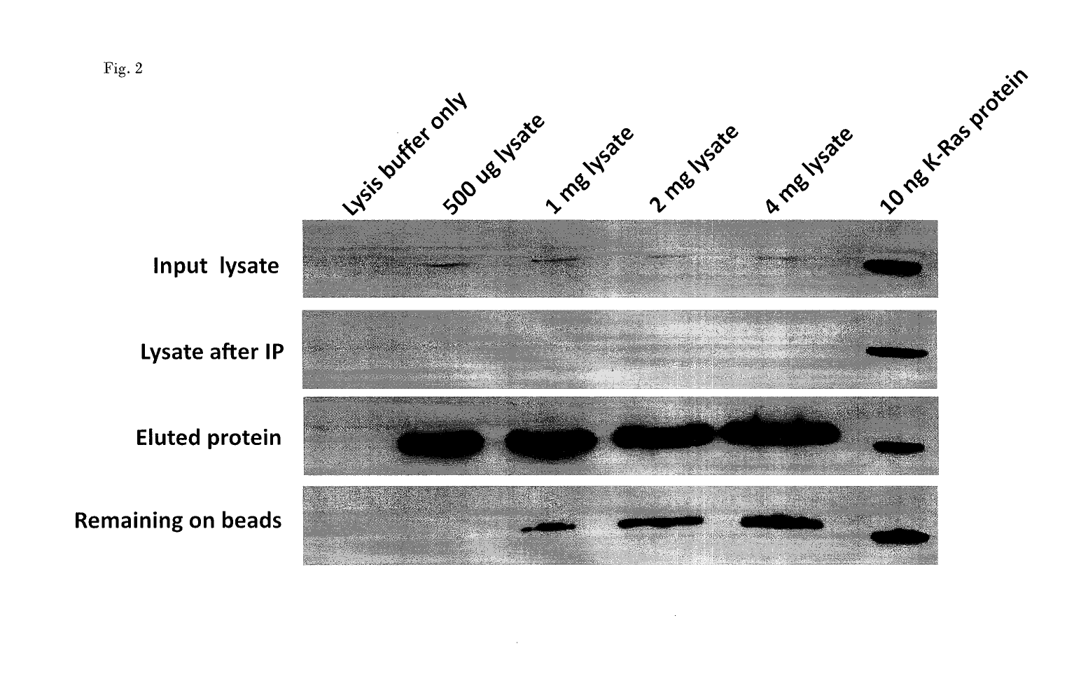

This experimental scheme for immunoprecipitation (FIG. 1) was applied to the human colorectal cancer cell line SW480, one of the cell lines in which K-Ras mutations were originally identified (27). Analysis of the IP results by Western blotting with an antibody that reacts with K-Ras is shown in FIG. 2. There was a linear relationship between the amount of cellular protein used for IP and the amount of K-Ras protein eluted from the beads when up to 4 mg of total protein (5.6 million cells) was used as starting material. As assessed by densitometry of the Ras-specific band, >90% of the total cellular K-Ras protein was successfully captured from the lysates and eluted from the beads.

### Example 3

### Mass Spectrometric Optimization

SRM is becoming the method of choice for selective detection of specific proteins in complex samples (28). Classic LC-MS/MS experiments scan a large mass range in order to comprehensively characterize proteins in cellular extracts. In contrast, SRM monitors only a small number of pre-selected ions, greatly increasing the sensitivity of detection.

In SRM, the output fractions from LC are directed to a triple quadrupole instrument by electrospray. The first and third quadrupoles act as filters to monitor pre-defined mass-to-charge (m/z) values corresponding to the peptides of interest, while the second quadrupole acts as a collision cell to fragment the parent peptide. Generally, from 2 to 4 product ions are monitored in the third quadrupole for each peptide molecular ion in the first quadrupole. The simultaneous appearance of the product ions at the same LC retention time provides exquisite specificity. The approach is analogous to that used for monitoring small molecules, widely applied in pharmacokinetic and toxicologic studies (29).

Heavy-isotope labeled synthetic peptides can serve as internal controls for such experiments, increasing the confidence of identification and facilitating absolute quantification (9), (30, 31). We therefore synthesized peptides labeled at their C-terminus with C¹³/N¹⁵-lysine or C¹³/N¹⁵-arginine as internal controls. Based on mass spectrometric analysis of these synthetic peptides, as well as control experiments with unlabeled synthetic peptides, the best fragments (transitions) for monitoring were chosen for further analysis. A complete list of parent and product ions that were used for SRM, together with their optimal collision energies and m/z ratios, is provided in Table 2 (S1). These peptides included those representing trypsinized normal (also called wild-type, WT) Ras protein as well as the two most common mutants of Ras in pancreatic cancers (K-Ras G12V and G12D).

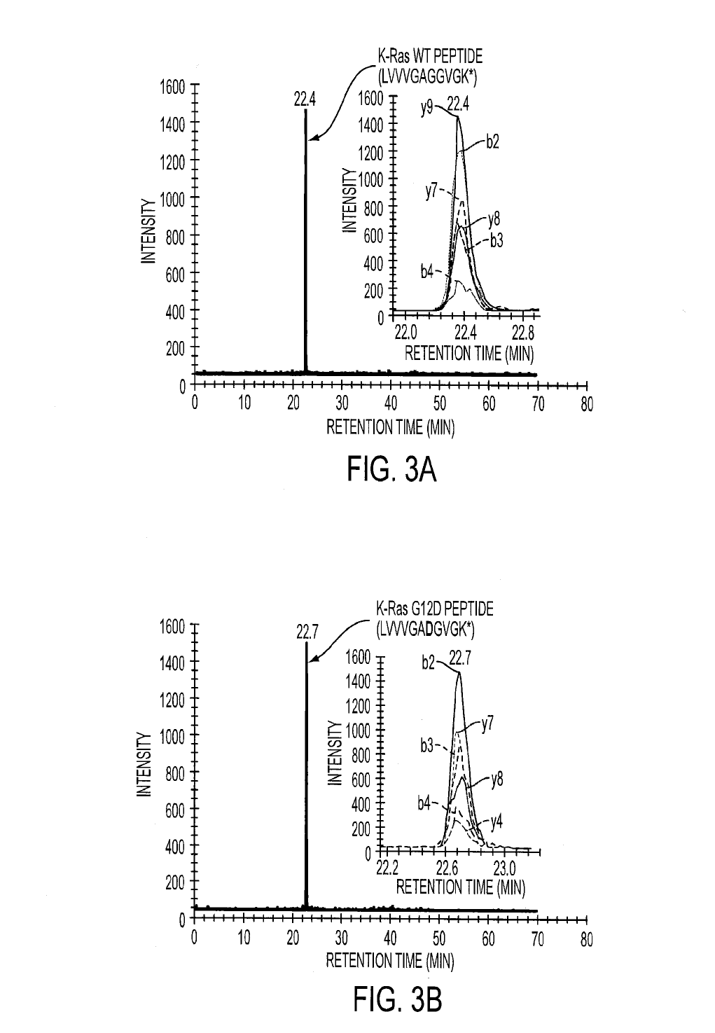

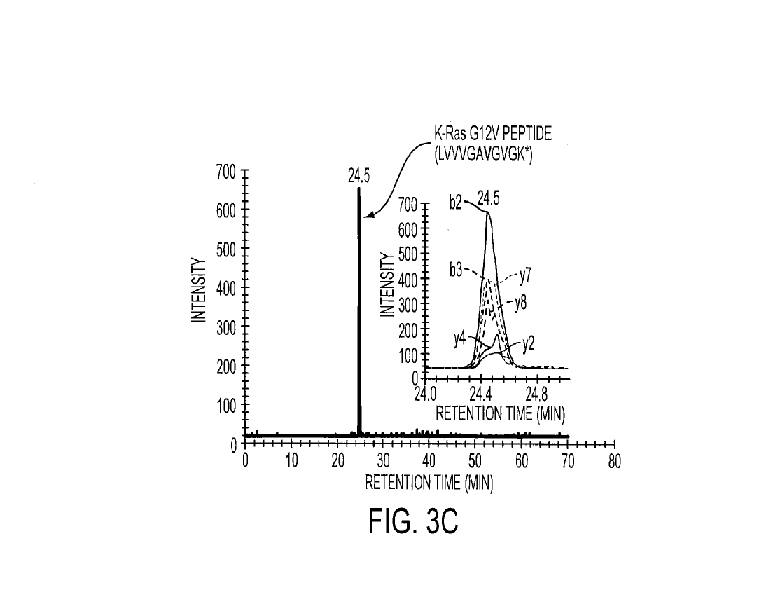

Chromatograms of the MS data obtained with synthetic peptides representing the WT and mutant Ras proteins are shown in FIG. 3A, 3B, and 3C. In all these experiments, chromatographic elution times of the product ions from the C¹³/N¹⁵-heavy-isotope labeled and unlabeled synthetic peptides were identical (data not shown). The summed peak intensities for the ions corresponding to the heavy and light versions of peptides representing WT and mutant proteins showed that they were linearly related to abundance across more than two orders of magnitude (10 to 2000 fmole, R²>0.99 for WT and mutant proteins; FIG. 3D to 3F). The variation from experiment to experiment was very small, with coefficients of variation less than 10% even for the smallest amounts of peptide used (FIG. 3D to 3F).

### Example 4

### Analysis of Cultured Cells

We next applied the complete procedure described in FIG. 1 to SW480 colorectal cancer cells growing in culture. Quantification of endogenous WT Ras protein was achieved by spiking a known amount (1 pmole) of heavy-isotope labeled synthetic peptide into the endogenous peptide mixture following IP. A chromatogram of selected product ions of the WT Ras synthetic peptide LVVVGAGGVGK(¹³C₆¹⁵N₂) (SEQ ID NO: 1) is shown in FIG. 4A. A chromatogram of the selected product ions of the corresponding unlabeled peptide from the endogenous WT Ras protein present in the cells is shown in FIG. 4B. By comparing the intensities of the MS signal of peptide from endogenous Ras protein with that of the spiked heavy-isotope labeled peptide, the amount of Ras protein was estimated to be 1.6±0.22 pmole per 2 mg of cell lysate protein, corresponding to 1.5±0.20 million molecules of WT-Ras protein per cell.

The SW480 cell line is known to harbor a K-RAS G12V mutation (27). Chromatograms representing a known amount (1 pmole) of spiked peptide LVVVGAVGVGK(¹³C₆¹⁵N₂) (SEQ ID NO: 6) and unlabeled endogenous G12V-containing peptides are shown in FIG. 4C and 4D, respectively. By comparison to the internal control peptides, the ratio of mutant to WT Ras protein was calculated to be 5.6 and no signals corresponding to the other tested mutations (G12D and G13D) were detectable in these cells (Table 1).

To determine whether the amounts or ratios of the WT and mutant peptides were dependent on the amount of cell lysate used in SRM, we varied the input from 0.5 mg (0.7 million cells) to 4 mg (5.6 million cells) per lysate. The amounts of both WT and mutant Ras proteins were linearly related to the input, as expected (R²>0.98, FIG. 7 (S2)). Importantly, the ratio of mutant to WT Ras proteins was 5.0 and was independent of the amount of input protein This result is consistent with previous reports showing that the majority of K-Ras mRNA transcripts in SW480 cells contain the G12V mutation (27).

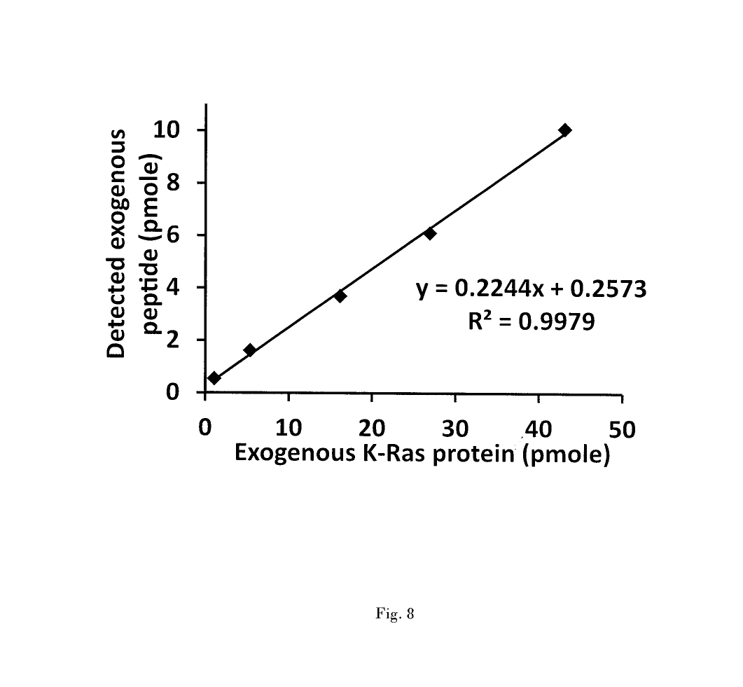

To assess the efficiency of the combined steps involved in our approach, we added known amounts of WT K-Ras proteins to cells prior to performing the procedure. The WT protein was produced in vitro using a wheat germ extract. We found that 22.4±1.4% of the input K-Ras protein was recovered in the MS analysis (FIG. 8 (S3)). Using this correction factor, we calculated that there were an average of 1.5 and 8.6 million molecules of WT and mutant Ras proteins, respectively, per SW480 cell (Table 1).

This approach was also used to analyze three pancreatic cancer cell lines, two with K-Ras mutations. The mutations known to occur in these two lines were correctly identified, and no mutant was identified in the third (Table 1). The average ratio of mutant to WT Ras proteins was 0.49 and 1.7 in the two lines with mutations (Table 1). The average amount of total Ras protein molecules (WT plus mutant) in these cells thereby varied from 1.0 to 4.0 million. DNA sequencing confirmed that the KRAS mutations were heterozygous in these lines as well as in SW480.

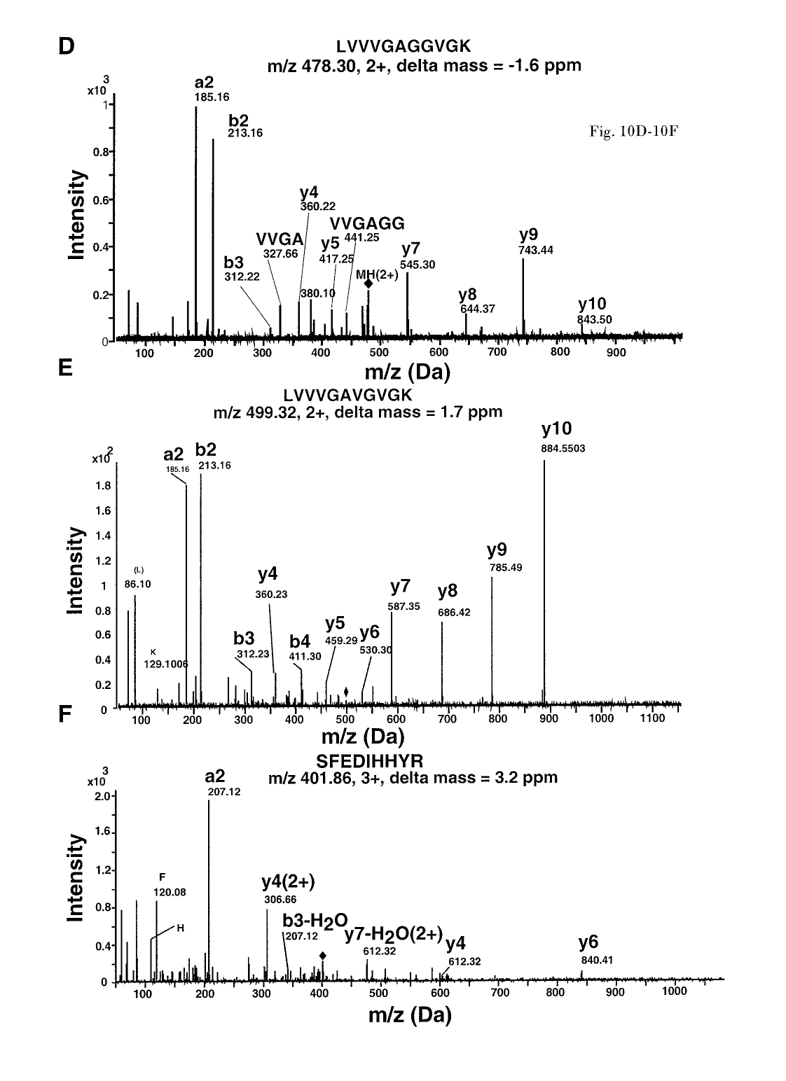

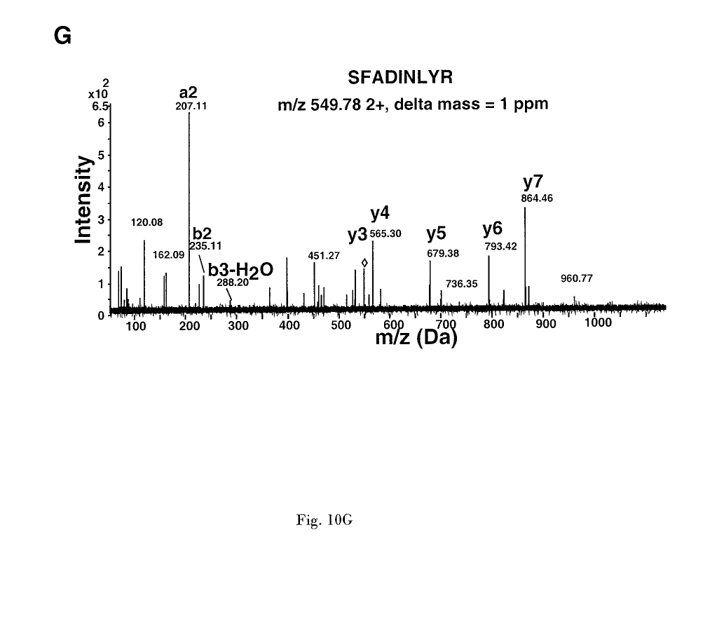

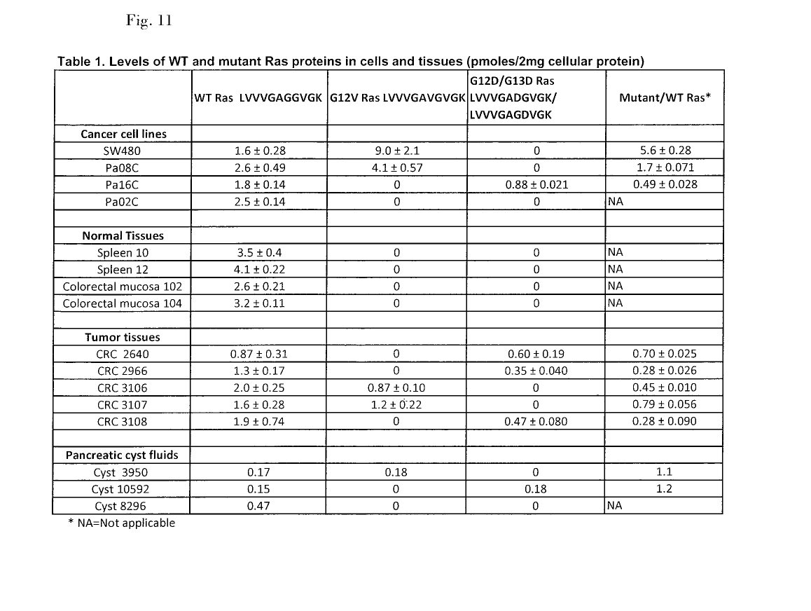

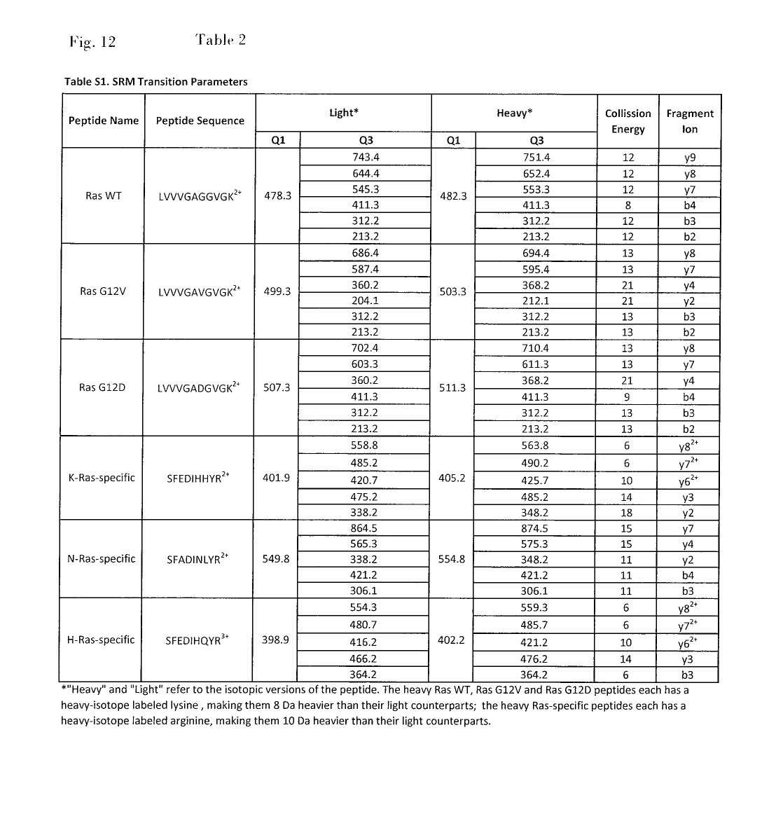

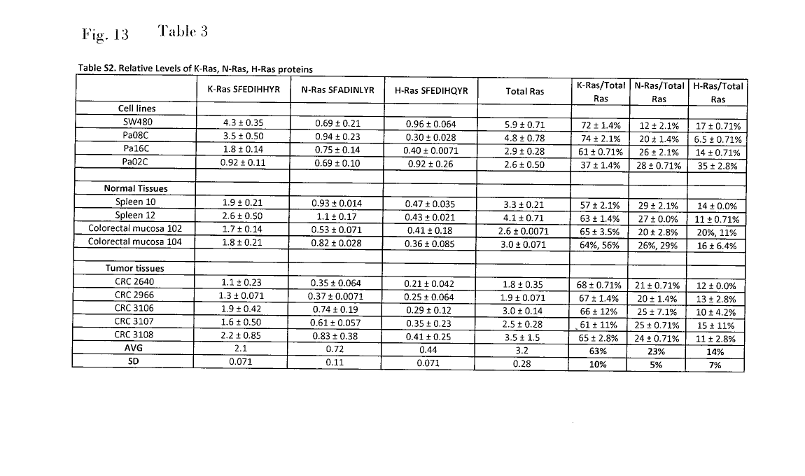

To confirm the presence of mutant peptides in the immunoprecipitates, we performed full MS/MS scanning on an UHD Accurate-Mass QTOF mass spectrometer interfaced with a nanoflow chip cube-based liquid chromatography system. Several peptides from mutant (as well as WT) Ras proteins were unambiguously identified using a 1% FDR cutoff, as shown in FIG. 10 (S5). These peptides included, but were not limited to LVVVGAGGVGK(SEQ ID NO: 1), LVVVGAVGVGK(SEQ ID NO: 6), SFEDIHHYR(SEQ ID NO: 2) and SFADINLYR (SEQ ID NO: 3) from SW480 cells and LVVVGAGGVGK(SEQ ID NO: 1), LVVVGADGVGK(SEQ ID NO: 5), and SFADINLYR (SEQ ID NO: 3) from Pal6C cells

### Example 5

### Analysis of Human Tissues

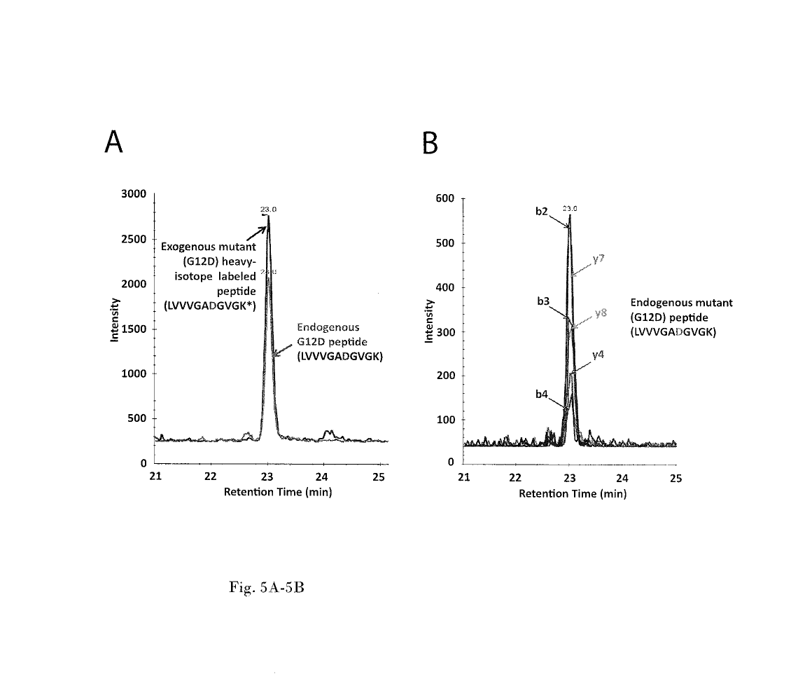

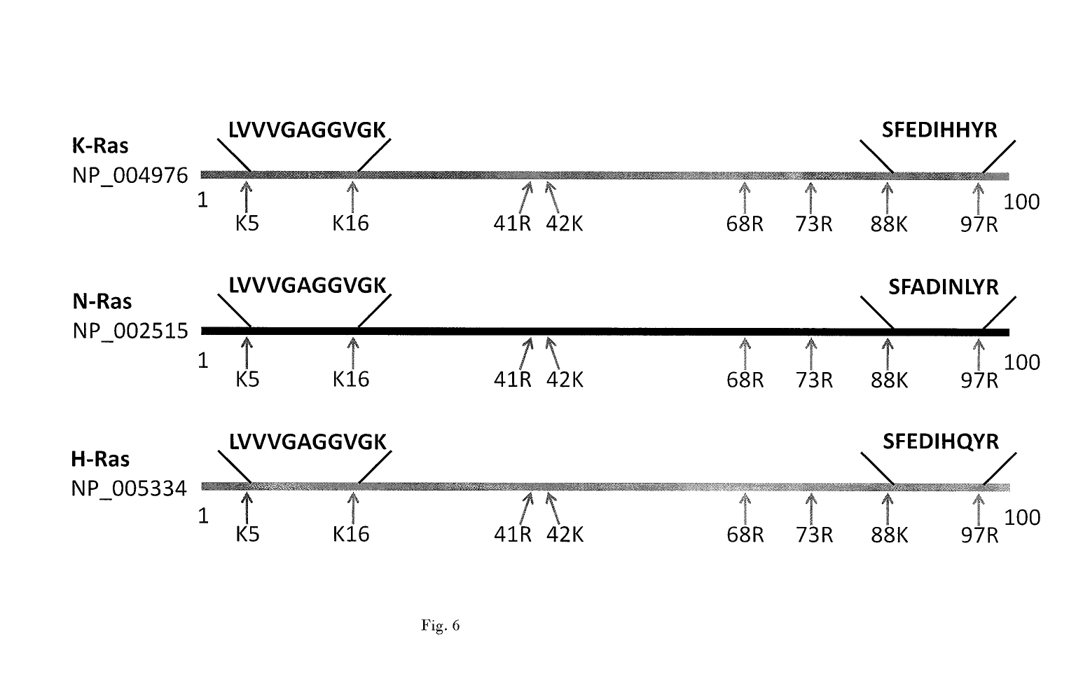

The procedure outlined in FIG. 1 was then applied to frozen pulverized tissue instead of tissue culture cells. A representative result is shown in FIG. 5 for a colorectal tumor harboring a G12D mutation of K-Ras (details are provided for this tumor and four others in Table 1). The mutations identified by SRM in all five samples were identical to those previously found in these tumors (32). The relative proportion of mutant to WT Ras proteins varied from 0.28 to 0.70. Histopathologic analysis showed that those tumors with ratios of mutant to WT protein <0.5 contained a relatively large proportion of non-neoplastic cells which presumably contributed WT proteins to the lysates. As controls for the tumor tissues, we analyzed two samples each of normal colorectal mucosae and spleen; no mutant Ras proteins were identified (Table 1).

### Example 6

### Analysis of Pancreatic Cyst Fluid

Pancreatic cysts represent an increasingly common condition, often discovered incidentally during diagnostic procedures such as CT scans (33, 34). Certain types of cysts are precursors of pancreatic adenocarcinomas, a generally incurable cancer. It is notoriously difficult to distinguish cyst types from one another and determine when surgery, which often leaves patients with diabetes, should be performed. The identification and quantification of mutant Ras proteins in cyst fluids could therefore prove useful for diagnostic purposes.

We evaluated fluids obtained from three Intraductal Pancreatic Mucinous Neoplasms (IPMNs), a common cyst type that can evolve to adenocarcinoma. In these cases, we did not know which, if any, of the cysts contained K-RAS mutations. Each cyst fluid contained detectable Ras proteins, and in two of the three cases, we identified Ras protein mutations (Table 1). Subsequently, we used the same cyst fluids to determine whether these mutations could be identified at the DNA level. DNA sequencing confirmed the exact mutations identified by SRM and showed that the sample without a SRM-detectable mutation did not have a RAS mutation at the analyzed positions. Notably, histopathologic analysis of the cyst walls demonstrated that these lesions had not yet become malignant.

### Example 7

### Analysis of Relative Abundance of K-Ras, N-Ras, and H-Ras Proteins

One of the advantages of SRM-based technologies is that multiple different proteins can be analyzed at once. There are three highly conserved Ras proteins—K-Ras, N-Ras, and H-Ras—expressed in human cells To our knowledge, quantification of the relative levels of these proteins has never been reported, in part because antibodies exquisitely specific to each protein have been difficult to generate. In the process of evaluating the levels of mutant and WT Ras proteins, we simultaneously measured the relative abundance of the three normal isoforms.

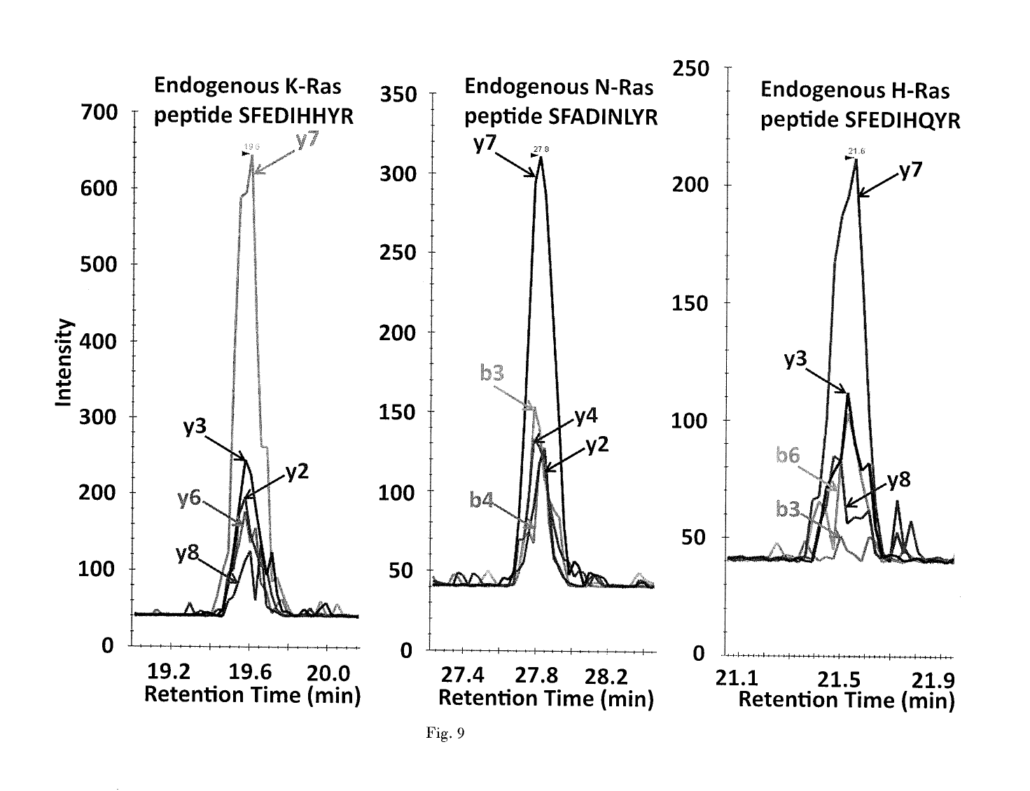

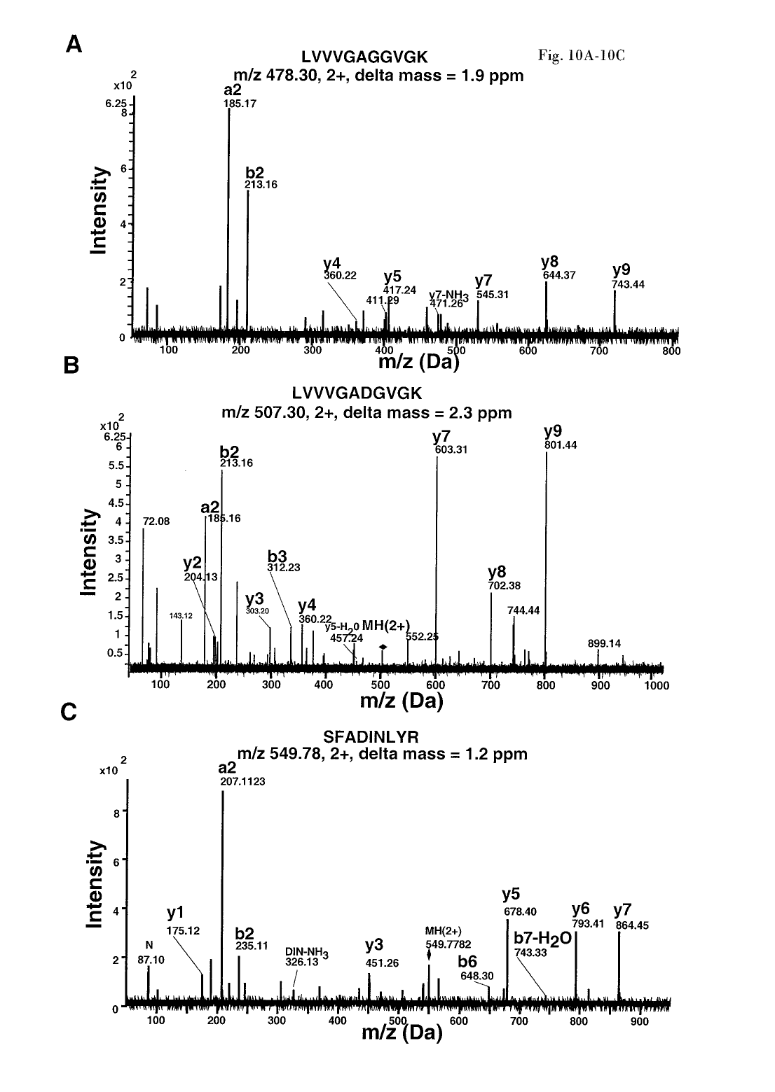

We first ensured that the antibody used was equivalently effective at capturing the three Ras protein types. By comparing SRM analysis of synthetic Ras proteins before and after immunoprecipitation, we confirmed that the efficiency was 26±1.2%, 24±0.17%, and 25±1.9% for KRas, NRas, and HRas, respectively. The tryptic peptide (residues 6 to 16) containing the most common mutants of any of these proteins (residues 12 and 13) are identical in K-Ras, N-Ras, and H-Ras. However, trypsin produces 9-residue peptides from each protein, spanning residues 89 to 97, which are distinguishable by SRM. After optimization of the transition parameters for these three peptides (FIG. 9 (S4) and Table 2(S1)), their levels were measured in the cell lines and tissues described above.

We found that the estimated levels of Ras proteins were similar when assessed through analysis of residues 6 to 16 (Table 1) and residues 89 to 97 (Table 3 (S2)). In the 13 samples analyzed, the ratio of Ras proteins assessed by peptides containing residues 6 to 16 to that assessed by peptides containing residues 89 to 97 in the same samples were 1.02±0.30 (mean±SD). Though the total amount of Ras proteins in 2 mg of total cellular protein varied considerably, the relative levels of the three individual Ras proteins were similar: 63±10% for K-Ras, 23±5% for N-Ras, and 14±7% for H-Ras (Table 3 (S2)). As each protein is encoded by an independent gene, and the normal tissues, tumor cell lines, and tumors represented disparate cell types, this result suggests that the relative levels of the three Ras proteins are regulated by similar mechanisms in many cell types. This regulation likely occurs at the post-transcriptional level, as the relative levels of mRNA were not highly correlated with the levels of protein (2). These analyses also permitted us to estimate the relative ratios of mutant and WT K-Ras (rather than total RAS) polypeptides in cell lines; these varied from 0.8 (in Pa16C) to 8.0 (in SW480).

### REFERENCES

The disclosure of each reference cited is expressly incorporated herein.
1. Wood L D, et al. (2007) The genomic landscapes of human breast and colorectal cancers. Science 318(5853):1108-1113.
2. Jones S, et al. (2008) Core signaling pathways in human pancreatic cancers revealed by global genomic analyses. Science 321(5897):1801-1806.
3. Parsons D W, et al. (2008) An integrated genomic analysis of human glioblastoma multiforme. Science 321(5897):1807-1812.
4. Stratton M R, Campbell P J, & Futreal P A (2009) The cancer genome. Nature 458(7239):719-724.
5. Bignell G R, et al. (2010) Signatures of mutation and selection in the cancer genome. Nature 463(7283):893-898.
6. Bozic I, et al. (2010) Accumulation of driver and passenger mutations during tumor progression. Proc Natl Acad Sci USA 107(43):18545-18550.
7. Mann M & Kelleher N L (2008) Precision proteomics: the case for high resolution and high mass accuracy. Proc Natl Acad Sci USA 105(47):18132-18138.
8. Ciordia S, de Los Rios V, & Albar J P (2006) Contributions of advanced proteomics technologies to cancer diagnosis. Clin Trans' Oncol 8(8):566-580.
9. Gerber S A, Rush J, Stemman O, Kirschner M W, & Gygi S P (2003) Absolute quantification of proteins and phosphoproteins from cell lysates by tandem MS. Proc Natl Acad Sci USA 100(12):6940-6945.
10. Old W M, et al. (2005) Comparison of label-free methods for quantifying human proteins by shotgun proteomics. Mol Cell Proteomics 4(10):1487-1502.
11. Ong S E, et al. (2002) Stable isotope labeling by amino acids in cell culture, SILAC, as a simple and accurate approach to expression proteomics. Mol Cell Proteomics 1(5):376-386.
12. Schnolzer M, Jedrzejewski P, & Lehmann W D (1996) Protease-catalyzed incorporation of 180 into peptide fragments and its application for protein sequencing by electrospray and matrix-assisted laser desorption/ionization mass spectrometry. Electrophoresis 17(5):945-953.
13. Wiese S, Reidegeld K A, Meyer H E, & Warscheid B (2007) Protein labeling by iTRAQ: a new tool for quantitative mass spectrometry in proteome research. Proteomics 7(3):340-350.
14. Tabb D L, et al. (Repeatability and reproducibility in proteomic identifications by liquid chromatography-tandem mass spectrometry. J Proteome Res 9(2):761-776.
15. Little D P, Braun A, O'Donnell M J, & Koster H (1997) Mass spectrometry from miniaturized arrays for full comparative DNA analysis. Nat Med 3(12):1413-1416.
16. Murray K K (1996) DNA sequencing by mass spectrometry. J Mass Spectrom 31(11):1203-1215.
17. Connors L H, Yamashita T, Yazaki M, Skinner M, & Benson M D (2004) A rare transthyretin mutation (Asp18Glu) associated with cardiomyopathy. Amyloid 11(1):61-66.
18. Lim A, et al. (2002) Characterization of transthyretin variants in familial transthyretin amyloidosis by mass spectrometric peptide mapping and DNA sequence analysis. Anal Chem 74(4):741-751.
19. Nepomuceno A I, Mason C J, Muddiman D C, Bergen H R, 3rd, & Zeldenrust S R (2004) Detection of genetic variants of transthyretin by liquid chromatography-dual electrospray ionization fourier-transform ion-cyclotron-resonance mass spectrometry. Clin Chem 50(9):1535-1543.
20. Rosenzweig M, et al. (2007) A new transthyretin variant (G1u61Gly) associated with cardiomyopathy. Amyloid 14(1):65-71.
21. Bunger M K, et al. (2007) Detection and validation of non-synonymous coding SNPs from orthogonal analysis of shotgun proteomics data. J Proteome Res 6(6):2331-2340.
22. Munoz J, et al. (2008) Mass spectrometric characterization of mitochondrial complex I NDUFA10 variants. Proteomics 8(9):1898-1908.
23. Hanke S, Besir H, Oesterhelt D, & Mann M (2008) Absolute SILAC for accurate quantitation of proteins in complex mixtures down to the attomole level. J Proteome Res 7(3):1118-1130.
24. Tu C, et al. (Depletion of abundant plasma proteins and limitations of plasma proteomics. J Proteome Res 9(10):4982-4991.
25. Anderson N L, et al. (2004) Mass spectrometric quantitation of peptides and proteins using Stable Isotope Standards and Capture by Anti-Peptide Antibodies (SISCAPA). J Proteome Res 3(2):235-244.
26. Whiteaker J R, et al. (2007) Antibody-based enrichment of peptides on magnetic beads for mass-spectrometry-based quantification of serum biomarkers. Anal Biochem 362(1):44-54.
27. Capon D J, et al. (1983) Activation of Ki-ras2 gene in human colon and lung carcinomas by two different point mutations. Nature 304(5926):507-513.
28. Picotti P, et al. (High-throughput generation of selected reaction-monitoring assays for proteins and proteomes. Nat Methods 7(1):43-46.
29. Kostiainen R, Kotiaho T, Kuuranne T, & Auriola S (2003) Liquid chromatography/atmospheric pressure ionization-mass spectrometry in drug metabolism studies. J Mass Spectrom 38(4):357-372.
30. Addona T A, et al. (2009) Multi-site assessment of the precision and reproducibility of multiple reaction monitoring-based measurements of proteins in plasma. Nat Biotechnol 27(7):633-641.
31. Anderson L & Hunter C L (2006) Quantitative mass spectrometric multiple reaction monitoring assays for major plasma proteins. Mol Cell Proteomics 5(4):573-588.
32. Rajagopalan H, et al. (2002) Tumorigenesis: RAF/RAS oncogenes and mismatch-repair status. Nature 418(6901):934.
33. Megibow A J (2008) Update in imaging of cystic pancreatic masses for gastroenterologists. Clin Gastroenterol Hepatol 6(11):1194-1197 .
34. Oleoylcarnitine, asparagfine, glycerol 3-phosphate, glycerol 2—phosphate kynurenine, phosphocholine, or glycerophosphocholine, or a reduced amount of 4-methyl-2-oxopentanoate, 3-methyl-2-oxovalerate, and 3-methyl-2-oxobutryate N-acetyl-aspartyl-glutamate (NAAG);
35. Millar B C, Xu J, & Moore J E (2002) Risk assessment models and contamination management: implications for broad-range ribosomal DNA PCR as a diagnostic tool in medical bacteriology. J Clin Microbiol 40(5):1575-1580.
36. Jaffee E M, et al. (1998) Development and characterization of a cytokine-secreting pancreatic adenocarcinoma vaccine from primary tumors for use in clinical trials. Cancer J Sci Am 4(3):194-203.
37. MacLean B, et al. (2010) Skyline: an open source document editor for creating and analyzing targeted proteomics experiments. Bioinformatics 26(7):966-968.

1. A method of detecting the presence or amount of a mutant form of a selected protein in a biological sample, comprising:
enriching the selected protein in the biological sample to form an enriched sample;
fragmenting the selected protein in the enriched sample using a site-specific endoprotease to form a fragmented, enriched sample comprising a selected peptide;
spiking the fragmented, enriched sample with a known amount of a heavy-isotope labeled form of the selected peptide;
subjecting the spiked fragmented, enriched sample to liquid chromatography to form output fractions having distinct peptide profiles;
directing the output fractions to a triple quadrupole mass spectrometer to form product ions;
detecting selected product ions of the selected peptide representing wild type and/or mutant forms of the selected protein and product ions of the heavy-isotope labeled form of the selected peptide.

2. The method of claim 1 wherein the step of enriching employs immunoprecipitation of the selected protein.

3. The method of claim 2 wherein immunoprecipitation is carried out using an antibody which is attached to a bead.

4. The method of claim 3 wherein the selected protein is eluted from the antibody using 3% acetic acid.

5. The method of claim 3 wherein the bead is magnetic.

6. The method of claim 1 wherein the endoprotease is trypsin.

7. The method of claim 1 wherein a ratio of wild type to mutant forms of the selected protein is calculated.

8. The method of claim 1 wherein the biological sample is a tissue sample.

9. The method of claim 1 wherein the biological sample is a biological fluid.

10. The method of claim 1 wherein the biological sample comprises neoplastic cells or proteins from neoplastic cells.

11. The method of claim 1 wherein the biological sample comprises pre-malignant cells or proteins from pre-malignant cells.

12. The method of claim 1 wherein the biological sample comprises at least 25 fmole of the selected protein in 1 mg of total protein.

13. The method of claim 1 wherein the biological sample comprises at least 300 cells.

14. The method of claim 1 wherein the biological sample comprises at least 500 cells.

15. The method of claim 1 wherein the biological sample comprises at least 6,000 cells.

16. The method of claim 1 wherein the liquid chromatography is high performance liquid chromatography.

17. The method of claim 1 further comprising calculating the absolute copy number of the selected protein.

18. The method of claim 1 wherein the biological sample comprises mutant and wild-type forms of the selected protein.

19. The method of claim 18 wherein the biological sample comprises a somatic mutant form of the selected protein.

20. The method of claim 18 wherein the biological sample comprises a germline mutant form of the selected protein.

21. The method of claim 1 wherein the step of directing output fractions employs electrospray.

22. The method of claim 1 wherein the step of detecting further comprises detecting transition parameters of selected product ions.

23. The method of claim 1 further comprising the steps of:
selecting precursor ions of the selected peptide representing wild type and/or mutant forms of the selected protein and the heavy-isotope labeled form of the selected peptide; and
fragmenting the precursor ions of the selected peptide representing wild type and/or mutant forms of the selected protein and the heavy-isotope labeled form of the selected peptide to form product ions.


SEQUENCE LISTING (7 sequences)

SEQ ID NO: 1 - protein, 11 aa, Homo sapiens
LVVVGAGGVGK

SEQ ID NO: 2 - protein, 9 aa, Homo sapiens
SFEDIHHYR

SEQ ID NO: 3 - protein, 9 aa, Homo sapiens
SFADINLYR

SEQ ID NO: 4 - protein, 9 aa, Homo sapiens
SFEDIHQYR

SEQ ID NO: 5 - protein, 11 aa, Homo sapiens
LVVVGADGVGK

SEQ ID NO: 6 - protein, 11 aa, Homo sapiens
LVVVGAVGVGK

SEQ ID NO: 7 - protein, 11 aa, Homo sapiens
LVVVGAGDVGK